(19) United States
(12) Patent Application Publication
(10) Pub. No.: US 2015/0297663 A1
(43) Pub. Date: Oct. 22, 2015

(54) Zoe's Divine Cream

(71) Applicant: Zoe Winifred Allen Bennett, Tallahassee, FL (US)

(72) Inventor: Zoe Winifred Allen Bennett, Tallahassee, FL (US)

(21) Appl. No.: 14/160,589

(22) Filed: Jan. 22, 2014

Related U.S. Application Data
(60) Provisional application No. 61/849,144, filed on Jan. 22, 2013.

(51) Int. Cl. A61K 36/886; A61K 36/33; A61K 47/44; A61K 47/46; A61K 31/59; A61K 31/355; A61K 36/185; A61K 9/00

(52) U.S. Cl. CPC A61K 36/886; A61K 36/185; A61K 36/33; A61K 9/0014; A61K 47/46; A61K 31/59; A61K 31/355; A61K 47/44

(57) ABSTRACT

The invention reveals a “Health, Wellness And Maintenance Product”, as well as the formula and mixture process for its preparation. The art of this invention is a “Health and Wellness Product”. Disclosing a Massage Cream found to contain many benefits to the human condition. Users boast of numerous benefits upon repeated use. The art of the invention positively benefits users, according to feedback received. It has been found beneficially soothing to the body. All benefits for this art have not been disclosed, and/or discovered, at this time. The invention promotes a “Healthier Body”. It's composition comprising Natural Herbal Product Components and Homeopathic Product Components. All of which are previously approved for human consumption, and or use.

The invention embodies and discloses a natural herbal plant composition and mixture which may soothe, ease, massage and improve the body's muscles, joints, burns, bruises, mild migraines, irritated skin, and enhances the natural beauty and texture of the skin. The invention also discloses the discovery that specific natural herbal compositions containing all natural active ingredients from herbal plants and/ or extracts in any and all forms, shapes, colors, scents and/or processes can work together as a topical mixture to be used on the body. The natural herbal mixture soothes, eases, massages and improves the body's muscles, joints, burns, cuts, bruises, mild migraines and helps enhance the body's natural beauty, texture on mammals including humans. Also included in this invention is the formula and mixture process that was invented, developed and created.

### SPECIFICATION

Ingredients Water (Aqua), Aloe (Aloe Vera), Theobroma (Cacao), Shea Butter, Prickly Pear Extract (Cactacea), Lanolin, Coconut Oil, Olive Oil, Jojoba Oil, Almond Oil, Tea Tree Oil, Vitamins D and E, Fragrance, (Perfume), and/or Cherry Extract of any kind, shape, form, color, scent, process (i.e. raw, powder, liquid, capsules, unscented etc.).

### Method

Step 1 Soften Theobroma (Cacao) butter to a maneuverable consistency (33.33% of total mixture)

Step 2 Soften the Shea Butter to a maneuverable consistency (33.33% of total mixture)

Step 3 Prepare Alcoholic Extract. Melt Pure Lanolin, mix with Alcohol once softened (5.55% of total mixture) separately place in a jar or container and set aside for later use.

Step 4 Prepare Prickly Pear Extract. Prepare Aqueous Extract in advance, and set aside for later use. First, prepare the fruit by washing prickly pear fruit (5.55% of the total mixture contains this extract which is placed in to composition (11.45% prickly pear and 5.55% purified water). Separately place in either a glass jar or a plastic container for later use.

Prickly Pear Extract will be prepared by thoroughly washing the entire fruit. Pealing the outer covering off of the fruit removes sticky spines. If you like, you can wash and then put the entire fruit into blender. Warm or Cold Water washing is performed to remove any bacteria which may be attached, as well as to clean the fruit. Wash the fruit in salted water to ensure destruction of any bacteria which may be present. The produce receives a bath or a soak into a salty water mixture. You can peel, or leave it whole, next place the cleaned fruit into your blender. Next place purified water into blender with approximately 1 to 2 prickly pears. Blend on “Puree Speed”. Add additional water if consistency is too thick. Aqueous Extract can be either a cold extract or a hot extract. If you boil the Aqueous Extract, you will destroy the majority of any bacteria which may be present.

Step 5 Prepare “Aloe Vera Extract” (Juice /Gel and/or Plant Extract if making from scratch and blend together) (8.33% of the total mixture)

Step 6 Mix “Essential Oils” and “Carrier Oils” into “Cream ‘In Oil ’In Water” Composition, mix “Fragrances” into mixture. Add “Cherry Extract” and “Fragrances of Choice” into the composition. (5.55% of mixture contains Essential Oils infused with fragrances, approximately ______ of fragrances either and/or Fragrance such as Lavender and Cherry Oil, Coconut Oil, etc. . . . )

### Combine All Ingredients Together Into Mixture

Step 8 Pour in additional “Aqua” (Purified Water, Preferably)

Step 9 Combine/mix ingredients together, mix and stir and mix and stir some more.

Step 10 Once all ingredients have been added, mixture will feel wonderfully smooth and luxuriating to the skin.

Step 11 Depending upon the existence of roughness in the user's skin texture, and upon intended use and or user, additional amounts of “Aqueous Extracts” consisting of “Aloe Extract”, and /or “Prickly Pear Extract” may be added to mixture. The extracts do make the skin look and feel better. Alcoholic Extract Lanolin and or Lanolin Extract, is included to bind oils and other ingredients.

The ingredients are mixed in order to blend them into a smooth consistency similar to cake batter. Using a few drops of the essential oils will loosen the mixture. Additional drops of oil may be added as required. It is easy to add too much oil. The blend should be smooth, however, direct sunlight with hot temperatures exposure to the sun, can liquefy the composition and it can become too liquid. If this should occur, you merely need to refrigerate the composition and it will stiffen up again. Cacao/Theobroma/Cocoa Butter, then Shea Butter the preferable order. Shea butter is a better lead base component when mixing lotions, or so this inventor has found.

### Zoe's Divine Cream Mixture Process and Ingredients

### Cream ‘in Oil’ in Water

Compositions derived from Cocoa butter, Shea butter, and oils, and water. This invention is limited to the use of purified water, and in combination with the above named ingredients and essential oil(s),which have various composition components, including but not limited to those identified herein. These components my be either active ingredients and/or in-active ingredients otherwise not disclosed. Inactive ingredients, can/may include ingredients necessary for the solubility of the cream components, but not active, therefore not necessarily necessary for inclusion, herein.

### Alcoholic Extract

Alcoholic Extract. The art of this invention embodies the use of an alcoholic extract an ingredient that may be derived and made from the combination of liquefied Lanolin and/or Alcohol, including but not limited to Isopropyl Alcohol. This alcoholic extract embodied in this invention is not limited to ny particular “type”, “brand”, “form” or “variation” of lanolin extract, included herein the art of this invention. The art of this invention, does not limit its sought protection, merely the use of generic lanolin and alcohol but also alcoholic extract however made. Plain alcohol is also included in the composition of this invention.

### Aqueous Extract

Aqueous Extract from Opuntia/Humifusa, Cactacea, including but not limited to plants identified as native in the state of Florida, including all members of the “Prickly Pear Cactus Family”, as it is better known globally. The art of this invention is expanded to include protection as to the ingredient and component aqueous extract as used in this invention. The aqueous extracts can be obtained from the whole plant of Aloe Vera, and/or Opuntia Humifusa, respectively, or from any part of the plants thereof (e.g., root, stem, leaf, or form or format). Further, as to this invention the extracts that can be used can not only be aqueous extracts but also alcoholic extracts, and/or cream in oil n water added to cream base/lotion base. As to this invention, any and all types of “Aqueous Extract” may be used and may be derived from any and all types of aloe vera plants in any form, including but not limited to liquid, gel, powder and/or any known or unknown format (presently known, or that may be discovered in the future). Aloe Vera, has been found to be a dilatant, it helps any composition to have a smoother consistency.

The “Inventor's Aqueous Extraction Process”, though unique to the inventor, is not limited to being a cold extraction process. It can/ and occasionally can/does entail incorporation of a heating process, boiling, and/or steam distilling. As stated the art of this invention is not limited to the preferred means, which is cold extraction. The composition can further include a “Moisturizing Agent”, a “Structuring” and/or “Thickening Agent”, not limited to any thrm, and/or brand. The resulting Art of this composition, can/may further include, a “Sun Screening Component”, and/or a “Sunscreen Agcnt”. The aqueous extracts, though not limited to, can be obtained from the Opuntia Humifusa and/or Cactacea plant family, and any member thereof, which has similar properties, and benefit by aqueous dilution. These processes, of similar result, from other known means of performing the extraction procedure, are also techniques which may yield the intent of the art of this invention, and are therefore intended to be covered under sought protections of this invention. There is no way to skirt around the intention of this inventor by variation of extracts or extraction process. Aqueous extracts can be obtained from, but are not/is not limited to all/or parts of the Prickly Pear Plant, though preferred to comprising, at least 90% from the fruit.

The art of this invention's composition noticeably relieves and/or eases stiffness, soreness and body aches due to its natural “Herbal Massage Composition”, and its intended capability to soothe. Further, said composition can be used to treat skin by topically applying said composition to skin, in need thereof. The art of this composition can be applied to the body, for topical use achieving both internal and external benefit. It can he applied to either normal skin, and/or to dry, flaky, sensitive skin, therein in need, thereof. The composition of the art of this invention can be applied to skin with wrinkles, therein in need of this composition. Thus, the composition the art of this invention, can be used to improve the health and wellness of the body and enhance the skin. There is no external limitation claimed for this topical composition, nor its intended use, nor the intent of the inventor for the composition of this art. New ways in which humans or mammals can benefit from this invention is currently ongoing and being identified daily, especially by users of the art of this invention. Users are merely applying, soothingly rubbing, and applying, massaging/some self applying, “Massaging Their Problems Away”, one rub down at a time, and one application, no more than 2 to 3 times daily.

In addition to the above combinations, the inventor of the art of this invention, has also discovered that a wide variety of plants, extracts, and included components, herein, have therapeutic benefits according to their known and established uses. Including all incorporated plants, extracts and oil combinations, associated to the art of this invention, its composition processes, and/or the specifications disclosed and/addressed herein. The combined natural ingredients enabled the creation of this herbal mixture which has significant benefit to the human body, without the use of dangerous pharmaceuticals.

In particular aspects, this invention embodies a composition, formula and process that is formulated as a topical skin composition. The composition includes a soothing and moisturizing agents. The invention can be prepared and made into a lotion, cream, a salve, and/or ointment, etc. . . . The compositions of the present invention can also include any one of, any combination of, and/or all of the following additional ingredients: water, a moisturizing agent, a preservative, a thickening agent, an essential oil, a structuring agent, a vitamin, and/or pharmaceutical ingredients, and/or any combination of such ingredients, or mixtures of such ingredients thereof necessary for the preservation of the invention.

The contents of the specification disclosed in the art of this invention. are incorporated herein by reference. It is contemplated that any embodiment discussed in this specification can be implemented with respect to any method and/or composition of the invention, and vice versa. Method(s) comprising topically applying the composition(s) of the present invention to body, skin, surface, in need thereof. By way of massage. Furthermore, compositions of the invention can be used to achieve methods of the invention.

### DESCRIPTION OF ILLUSTRATIVE EMBODIMENTS

Zoe's Divine Cream, or ZDC as it has been called, the art of this invention, is an effective alternative use of natural herbal compositions and ingredients currently used to soothe, massage, improve and enhance the body and skin. As noted above, particular combinations of the aforementioned/alluded to extracts have been discovered to work well with the human body and skin. The skin is the largest organ of the human and/or animal body. This inventor's inclusion/utilization of extracts disclosed in the specification have also been discovered to soothe, improve, ease and enhance numerous body and skin issues, without use of a pharmaceutical drug.

### Product Description

The art of this Invention embodies and comprises a “Health, Wellness and Maintenance Product” that can be considered a “ Natural ”, as well as “Herbal Product”, and/or a Homeopathic Product comprising of an all natural topical massage and body cream. The primary purpose of this natural product to help and provide those individuals who prefer and seek alternative natural products to pharmaceutical products that routinely tend to cause many allergic reactions as they contain strong and dangerous chemical drugs. Pharmaceutical products have both short and long term risks. This natural product invention and its active natural ingredients have been proven to have little to no allergic or detrimental affects to humans or mammals. This natural herbal skin cream is simply to make one's body and skin feel well, healthy, terrific and sooth and soften the body quickly. Users are encouraged to apply this natural product as needed during the day. Daily use for a period of time, will also help keep the skin moisturized. Using this natural product cream helps to soothe, eases, improves and massages joints, muscles, bruises, pain, minor headaches and migraine headaches in a safe and noninvasive but natural way.

### BACKGROUND OF INVENTION

A. Field of the Invention

The present invention embodies and comprise of compositions that include one or any combination of plants or extracts thereof selected from the group consisting of Aloe (Aloe Vera), Theobroma (Cacao), Shea Butter, Lanolin, Fragrance, (Parfum) Almond Oil, Cherry Extract, Coconut Oil, Jojoba Oil, Vitamin E, Olive Oil, Tea Tree Oil, Water (Aqua) and Prickly Pear Extract (Cactacease) of any kind, form, texture, shape, color, process, scent, unscented etc. The compositions can be formulated as topical skin compositions.

B. Description of Related Art

This art of this invention embodies and comprised, at least of, natural herbal ingredients consisting of (a) Aloe Vera Extract(s)(ed) from the plant—Active Ingredient, (b) Shea Butter (derived from plant or tree- Active Ingredient), (c) Cacao Butter (Cocoa Butter) (derived from Theobroma Nut Plant or Tree—Active Ingredient), (d) Lanolin Extract (derived using Isopropyl Alcohol and Lanolin—Active Ingredient), (e) Extract from Opuntia Cactacea, including but not limited to Opuntia Humifusa—(and/or any other variation and/or part of the Prickly Pear Cactus family)—Active Ingredient, (f) Extract from Cherry fruit (derived from the fruit from the tree)—Inactive ingredient, (g) Fragrances-Inactive ingredients, including but not limited to Coconut fragrance and /or any variation including Coconut Oil,—Active Ingredient, (h) Essential oils, including but not limited to Jojoba Oil, (i) Tea Tree Oil,—Active ingredient, etc. . . . , and finally, (j) Vitamin E,—Inactive ingredient, not limited, of course. The resulting composition(s) can be formulated including but not limited to cithcr, topical skin compositions, creams, lotions, emulsions, and/or salves and/or any variations of said components, so described herein.

The present invention specifically relates to compositions, that include combinations of plants, and/or extracts thereof, selected from the group comprised at least of, consisting of, or comprising of: Herbal plants identified in previous sections of this application, including the use of inactive ingredients necessary to prevent spoilage and/or contamination. Inactive ingredients, necessary for use as preservatives, or thickeners including, but not limited to the likes of essential oils, antimicrobials. such as say, tea tree oils, carrier oils, etc. . . . , thickeners and or preservatives for example glycerin, which all of which, or/any combination thereof, which may be either actively or inactively included into said composition.

The invention discloses natural herbal plant composition(s) and mixtures which have been stated to quickly soothe, ease and improve the body, muscles and/or joints, upon application. The mixture has been described as providing benefit to the youthfulness of skin. This natural herbal composition can be administered and/or used on mammals, including but not limited to humans, not limited, of course. Also, included in this invention's protection, is/are the selected component ingredients incorporated into this mixing process in any shape, form, format, texture and/or type, process stage, raw, unprocessed, virgin, extra virgin, etc. . . . The recipe/and/or method, of which the art of this invention was created, in order to compose, and mix together select natural herbal ingredients, that would effectively function, and/or work together for the benefit of the body, not limited to the skin, even though, application is topically intended.

The art of this invention addresses several complaints about how the body changes as humans age by using this invention by way of body massage and/or application of cream/lotion/ointment/salve/emulsion/sunscreen/Sunblock/etc. . . . However, THIS ART DOES NOT EXCLUDE OTHER USER IDENTIFIED BENEFITS OF NATURAL HERBAL INGREDIENTS, NOT HEREIN DISCLOSED, BUT INCORPORATES THEM BY REFERENCE OF FAMILIAR USES OF DISCLOSED ACTIVE INGREDIENTS. Users of this invention claim that this invention, soothes and eases away certain types of aches, pain including but not limited joint and muscle pain and mild migraine headaches.

We can all benefit from the art of this invention, as time waits for no one. When we age, measureable changes occur in the body, and within the skin. Damage is not, nor should it be, limited to, loosing moisture or loosing youthfulness of appearance. or the ability to function as usual. Additionally, as the body ages, gravity and/or deterioration causes changes in the appearance of the body. In all of its former glory, the body may require pampering, lubricating, massaging, and or special treatment, in order to be refreshed, replenished, and/or rejuvenated. This composition, its natural herbal components, their beneficial properties, coupled with the identified natural and herbal ingredients herein, provides surprising results.

### SUMMARY OF INVENTION

The inventor discovered that certain types of ingredients when combined and mixed together can be used to soothe, ease, massage and improve the body. Prolonged use of this invention has been observed to result in improving the muscles, joints, pain, headaches and mild migraine headaches and enhancing skin tone, texture and coloring. There has been discovered, that this natural product with its all active herbal ingredients and compositions, have similar effects as reducing inflammation. The First composition ingredient component is an aqueous extract from Opuntia Humifusa, a plant that has been identified as native to Florida, and/or being a member of the Prickly Pear Cactus family, as it is better known globally. A second component ingredient is an alcoholic extract derived from the combination of lanolin and/or isolpropyl alcohol. The final composition can then be an emulsion, a cream, or a lotion aqueous extract from Aloe Vera plant. The composition can further include a moisturization agent, a structuring and/or thickening agent, not limited to glycerin, and/or an emulsifier.

The aqueous extracts can be obtained from the whole plant of Aloe Vera, Opuntia Humifusa, respectively, or from any part of the plant thereof (e.g., root, stem, leaf). Further, and in addition to the extracts being aqueous extracts, the extracts can also be alcoholic extracts, glycolic extracts, and/or oil extracts. The composition can be a beautification product, a skin toner/toning product, and /or a skin moisturizing product. Furthcr, said composition can be used to treat skin by topically applying the composition to skin in need thereof, wherein topical application of said composition treats said skin. The composition can be applied to aging skin and/or wrinkling skin. The composition can be applied to irritated or inflamed skin and/or dry skin, and/or flaky skin.

The composition can be an emulsion, a cream, a lotion, and/or a salve. Aqueous extract from Opuntia Humifusa. The composition can further include a moisturization agent, an antioxidant, a structuring or thickening agent, and/or an emulsifier (examples of each of these ingredients is provided below). The composition can. Aqueous extracts can be obtained from the Opuntia Humifusa plant, the extracts can be obtained from the fruit of the cactus plant, but is not limited to such. Other aqueous extracts can be obtained from the whole plant of Aloe Vera. Further, and in addition to the extracts being aqueous extracts, the extracts can also be alcoholic extracts, glycolic extracts, and oil extracts. Said extracts are obtainable from adding incrementally to cream base. The composition can be a skin beautification product, a skin toning product, and/or a moisturizing product. The composition can noticeably reduce inflammation from sore and inflamed joints, tissue and body parts such as inflamed joints. Further, said composition can be used to treat skin in need thereof by topically applying said composition to skin. The composition can inhibit inflammation activity in said skin. As such, the composition can be applied to inflamed skin, dry skin, flaky skin, and/or even to sensitive skin. The composition makes skin and body, look and better.

This invention of natural herbal composition can be a skin beautification and enhancement product, a skin toning product Of a moisturizing product. Said composition can also be used to treat skin by topically applying the composition to skin, in need thereof.

The composition can be a lotion, cream, emulsion or salve. The compositions of the present invention can also include, but is not limited to anv combination thereof, of and/or all of the following additional ingredients: water, a moisturizing agent, a preservative, a thickening agent, essential oils, a structuring agent, vitamins, pharmaceutical ingredients, and/or any combination of such ingredients, or mixtures of such ingredients thereof.

In another embodiment. there is disclosed a topical skin care composition that includes one and/or any combination thereof, the aforementioned plants, plant parts, and/or extracts thereof mentioned in this claim documentation, in combination with any one of, any combination of, or all of the following ingredients: water; glycerin; In particular aspects, the composition can further include any one of, any combination of, or all of the following additional ingredients: alcohol; isopropyl alcohol; and glycerin. The concentrations of these ingredients are not being delineated by weight and/or volume. In particular aspects, the concentration of water will be/can be determined based on quantity mixed/measured for production (in other words dependant of whether we are producing a little or a lot at any particular time.

In another embodiment, there is disclosed a topical skin care composition that includes as one of or combination of the aforementioned plants, plant parts, and/or extracts thereof in combination with any one of, any combination of, or all of the following ingredients: water; glycerin;

In another embodiment, there is disclosed a topical skin care composition that includes any one of or combination of the aforementioned plants, plant parts, and/or extracts thereof in combination with any one of, any combination of, and/or all of the following ingredients: water; glycerin; petrolatum; The composition can be a topical skin composition. The extracts can he aqueous, and/ or non-aqueous extract. The aqueous extracts) can include an alcohol, a glycol, and/or water.

One aspect of the present invention concerns a method of treating or preventing a skin condition comprising topically applying any one of the compositions disclosed in this specification to skin. In one embodiment of the present invention there is disclosed a method of reducing the appearance of symptoms associated with erythema (e.g., irritated skin, sensitive skin, inflamed skin tissue or joints). Method comprising topically applying the composition(s) of the present invention to body, skin, surface, in need thereof. Erythema can he caused by skin sunburn, skin burns, exercise, insect bites, bacterial infection, viral infection, fungal infection, massage, etc. As noted throughout, the composition can include a plant, plant parts, or extract(s) thereof.

In one particular non-limiting embodiment, the extract or extracts used in any of the treatment methods described above and throughout this specification and claims is prepared in accordance with the procedures described in the specification. Therefore, said contents or specification, are incorporated herein by reference. It is contemplated that any embodiment discussed in the specification can be implemented with respect to any method and/or composition of the invention, and vice versa. Furthermore, any compositions of the invention can be used to achieve methods of the invention.

In one embodiment, the topical skin compositions of the current invention are pharmaceutically elegant. Term describes compositions that have tactile properties which when applied to skin, feel pleasant on the skin. And in other words, compositions that are not too watery and/or greasy; compositions that have silky texture; non-tacky or sticky compositions. This term also can relate to moisture retaining properties of the invention.

“Topical Application” refers to applying composition onto the skin surface. Is suitable for topical application, solely. Other objects, features and advantages of the present invention will become apparent from the following detailed description. Additionally, it is contemplated that changes and modifications within the spirit and scope of the invention will become

### Plants, Extracts and Essential Oils Herein is provided below:

### OPUNTIA HUMTFUSA and All Prickly Pear Cactus, Cactacea

### Active Ingredient

Prickly Pear, is the commonly known name. Cactus have been found in numerous regions around the world, especially in the Western Hemisphere. Most people are familiar with the fact that cactuses grow in the desert. But it is little known that certain types are native in Florida. Florida has 9 species, in that sunshine state. They have been identified in counties around the state. They are grown commercially around the world. The entire plant can be edible. They are known as Sticky Succulents and well known as burglar prevention plants.

Opuntia Humifusa is a genus of the cactus family Prickly Pears. Florida boasts 9 PRICKLY Pear Species. The inventor has discovered that extracts of Opuntia Humifusa have several biological activities extremely beneficial to the human body, including but not limited to the outer layers of the human skin.

### ALOE VERA PLANT, All Types and in Non Limited Forms (ACTIVE INGREDIENT)

Aloe Vera Plant, known to reach 6 to 15 meters in height. Its leaves are 4 to u centimeters long, and are capable of producing seeds, flowers and fruit. Aloe is native of both Asia and Europe. Practically everyone on the planet knows that Aloe is very beneficial to the human body. The Aloe plant has biological activities which can benefit the skin and the body, Not Limited. Aloe Vera, its both edible and ingestible.

### SHEA BUTTER (ACTIVE INGREDIENT)

Shea butter is derived from the Shea Nut Tree, also known as the Karite Tree, or Butryospermum Parkii

### Meaning:

Parkii is known as the tree of life.

The part of the plant used in making Shea butter—the hard kernel inside the hull. This inventor's research disclosed that fresh or green fruit, makes the most potent and nutrient enriched versions of Shea Butter. The ones that have not turned because they are still green. Accordingly, older fruit is less potent and not as beneficial for the intention of this art.

### Contains:

i. Vitamins A

ii. Vitamins E and

iii. Vitamins F

iv. Fatty Acids and

v. Antioxidants respectively

### Uses:

i. Improves Skin Conditions

ii. Wrinkles

iii. Rashes

iv. Sunburns

v. Blemishes

vi. Scars

vii. Psoriasis (allegedly)

viii. Exema

ix. Anti-Aging and

x. Provides benefit for Anti Free Radicals

### Method of Extraction:

i. Processed by extracting the fruit, crushing and then boiling it.

ii. Nuts are picked

iii. Cracked, and

iv. Pounded Just to get the butter

The fruit solid that is removed is boiled in water until what is known as Shea butter rises to the top.
a. Shea butter can be extracted by “Pressing” that an expeller type press. Shortening the processing time by approximately 2 to 5 hours.
b. No Chemicals are required in order to obtain “Raw” or

“Unrefined” Shea

### Benefits:

i. Skin

ii. Circulation

iii. Dry Skin

iv. Moisturization with no greasy residue, is enriched with oil, provides youthful appearance

### Mineral Oil (White) (in Active Ingredient)

i. NF Food grade

ii. Great for a number of uses.

### Jojoba Oil

i. 100% pure:,

ii. Known for its high vitamin E content gives it an almost indefinite shelf life.

iii. Is an Essential Oil dilatant—

iv. Encarta defined “Dilantant” as—a substance that causes another to expand

v. Offers Moisturizing Properties making it:
a. Ideal For Massage Oils
b. Ideal for Lotions
c. Ideal for Lip Balms
d. Ideal for Soaps, Etc. . . .

### Benefits and Uses:

i. Moisturizes Cuticles

ii. Won't Clog Pores And Cause Acne

iii. Can be used As An Emollient.

iv. Treats acne.
v. Unclog Hair Follicles Encouraging The Re-Growth Of Thinning Hair.
vi. Removing makeup.
vii. Conditioning eyelashes.
viii. Add to soap moisturizer with oil. Resembling oils produced naturally in the human body.
ix. Infuse with your favorite essential oils to customize a personalized fragrance, will last for years.

### History of Jojoba Oil:

i. Produced From Jojoba Plant Seed.

ii. Technically A Liquid Wax.

iii. Native to the Mojave Desert.

iv. First commercially grown to provide a substitute for sperm whale oil in 1980. Prevented extinction and drought.

v. Besides helping prevent the demise of one of nature's most magnificent creatures, the Jojoba shrub is used to prevent dry regions from desertification by supporting vegetation and wildlife.

vi. Indigenous to US and Mexico.

vii. Grown now in Argentina, Australia, Israel, Peru and Palestine are beginning to harvest the benefits of this drought resistant shrub.

viii. Contains No Triglycerides.

ix. Has a long shelf life.

x. Has Natural Benefits.

xi. Has many Skin Nourishing Benefits.

xii. Great For Use In Lotions, Moisturizers. Shampoos, Conditioners, Soap Making, and as carrier oil for perfumes.

xiii. Medicinal Uses And Benefits.

For Health Vitality:

i. Is similar to the natural oils in human skin,

ii. Is Absorbed Quickly without that Greasy Feel.

iii. Moisturizes Face.

iv. Prevents “Shiny” Look.

v. Trains Skin To Produce Less Oils In Certain areas.

vi. Eliminates Need For Thick Lavers Of Make-Up. Which can irritate sensitive skin.

vii. Repeat use Shrinks Size Of Your Pores.

viii. Nearly Hypoallergenic.

ix. Used to Prevent Skin Infections From Acne, Insect Bites And Fungal Infections.

x. Effectively Treats Acute Skin Conditions Like Psoriasis, Eczema And Rosacea, Without Harsh Prescription Drugs.

xi. Used as A Carrier Oil

xii. A Favorite Essential Oil For Aromatherapy

xiii. Benefits The Mind, Mood And Cognitive Function

xiv. Is Non-Digestible In Mammals. Due to its ester like molecular structure.

xv. A Laxative Treating Constipation.

### For Hair and Skin Care:

i. Commercial cleansers use manmade ingredients aimed at cleansing without stripping away too much moisture.

ii. Natural components enhance natural beauty.

iii. Jojoba oil along with other natural oils make a cleansing and skin protecting composition.

iv. Not drying and/or irritating.

v. Use and rejuvenates the natural as a lotion replenishes oils in your skin.

vi. Apply to areas prone to dryness.

vii. Use before using sun tanning products ensures uniform results.

viii. A hot oil treatment that works to prevent split ends.

ix. Adds shine and luster to hair.

x. Relieves frizz.

xi. Should be used in dilution, and

xii. Should be avoided during pregnancy.

Viii. Is considered toxic if taken internally.

xiv. Is a non-irritant.

xv. Is non-sensitive.

xvi. Avoid if you have high blood pressure, and/or epilepsy.

### Pure Coconut Oil (Inactive Ingredient and an Active Ingredient

i. Our 100% pure Coconut oil

ii. RBD comes from the ‘cocos nucifera’ or coconut palm.

iii. Is expeller pressed oil extracted from the actual ripened coconut.

iv. Rich in medium chain fatty acids (found in breast milk.)

v. Is a vital ingredient in many foods.

vi. Is used for medicinal,

vii. Is used for bath and body purposes

### Most Common Uses of Coconut Oil:

i. It used as Fuel.

ii. In terms of Health And Beauty, Used As A Moisturizer In Dry Skin,

iii. Strengthens Hair.

iv. A Lubricant

v. A main ingredient in Soap Making.

vi. Refer Material Safety Data Sheets (MSDS) IN APPENDIX

### History of Coconut Oil:

i. Global Staple

ii. Known As the “Tree Of Life” by pacific islanders, thought to be a cure all.

iii. 2 to three 3 thousand years ago it the oldest traditional crop grown.

### Uses and Benefits:

### Soap Making:

i. Lauric Acid and

ii. Solid At At Temperatures below 76° F.,

iii. the single most popular oil in cold and hot process soap making

iv. Produces a Thick Lather.

v. This product is All Natural.

vi. Product is Cold Pressed and

vii. Hexane Extracted.

viii. Coconut oil helps soap retain moisture

ix. Hardening and whitening the finished product.

### Natural and Healthy Cooking:

i. Made from the dried coconut kernel.

ii. An excellent addition to a healthy home menu.

iii. Coconut oil is very heat stable, which means that it is great for high temperature cooking, like sauteing or flying.

iv. Slow to oxidise and resists rancidity, a long shelf life without getting that “stale” taste.

v. A Saturated Fat

vi. Completely trans-fat free

vii. Promoting Good Cholesterol

viii. A Natural Antibiotic And Aids In Nutrient Absorption

### A Healthy Complexion:

i. Healthy bodies start on the inside.

ii. A complete approach to the entire body and what it requires is the complete approach. We begin with the skin. Which is the largest organ of the body.

iii. Coconut oil effectively improves skin hydration,

iv. Coconut oil increases skin lipid levels,

v. Coconut oil has no irritant effects.

vi. Natural moisturizer rubs in easily,

vii. Doesn't leave an “oily feel”.

viii. Coconut oil has become a staple for the personal care industry.

### Provides Moisture to Dry Skin.

### GLYCERIN (INACTIVE INGREDIENT)

i. Known as “VG”.

ii. Kosher food grade USP Vegetable Glycerin has countless uses.

iii. In fact, there is only one producer lett in the USA for high quality glycerin production.

iv. Sensitive Pharmaceuticals are the only current market for this product.

v. Glycerin food industry.

vi. Personal care

Extraction Techniques: Extraction Not Limited to the following:

i. Aqueous-Extracts,

ii. Alcoholic Extracts.

iii. Cream in Water in Oil Components.

All those with prior familiarity due to Practical Exposure and/or Experience in the art of this type of invention would be capable of creating and/or recreating the embodiment of the art of this invention.

### Composition(s) of the Present Invention

i. Combinations and Amounts of Ingredients

### Composition Vehicles

The composition of the present invention, ZDC, can be formulated into all types of vehicles, including but not limited to Creams, Lotions, Emulsions, and/or Salves, roll on and liquids. In some aspects, the concentrations and combinations of ingredients can be selected in such way that the combinations are compatible, and do not complicate or deviate from ZDC's intended product. The plant extracts and additional ingredients identified throughout the specification are only intended for TOPICAL APPLICATION for targeted areas. In other words, this inventor merely intends that application be made only for areas, in need, thereof. Topical application to areas such as massaging/rubbing composition of this art over “the Body”, “the Joints”, or “the Skin”. Benefits claimed are Not Limited to those disclosed here in this claims protection. However, the language of “Not Limited to those disclosed”, is now incorporated by reference, herein.

### Products

The composition is of the art of this invention, Zoc's Divine Cream, or ZDC as it is abbreviated. The art of this invention, embodies a “Natural Herbal Health Wellness Maintenance Product”, it is considered by the inventor of this art as a Health and Wellness Natural Herbal Product due to its health and beautification results. As well as the identified and globally known beneficial uses of both active and inactive ingredient components.

### Cosmetic Ingredients

Sunscreen Agents; Moisturizing Agents: Structuring Agents; Essential Oils; Thickening Agents;

### Pharmaceutical Ingredients

Any plant derived preparation could be considered a pharmaceutical ingredient due to its propensity to be a healing agent.

The invention claimed is:

1. A topical skin composition comprising of: (1) herbs from aloe vera, shea nut butter, cocoa butter and cactus plants, (2) alcohol, (3) aqueous extract from cactacea opuntia, (4) cherry or coconut tlavoring, (5) lanolin extract, (6) almond, coconut, jojoba, olive, Lea tree oils and (7) vitamin D and vitamin E

2. The topical skin composition of claim 1, wherein the composition is a cream.

3. The topical skin composition of claim 1, wherein the composition is a salve, lotion, solution, roll on, gel, ointment or solution.

4. The topical skin composition of claim 3, wherein the composition comprises a moisturizing agent, a structuring or thickening agent, and an emulsifier.

5. The topical skin composition of claim 3, wherein the composition includes about 33.33% cacao, 33.33% shea butter, 5.55% alcoholic extract, 5.55%, prickly pear extract, 8.33% aloe vera extract, 5.55% essential oils, carrier oils, alcohol 2.77% and 0.05 fragrance.

6. The topical skin composition of claim 4, wherein the composition further includes a sunscreen agent.

7. The topical skin composition of claim 1, wherein each of said extracts are plant or alcoholic extracts.

8. The topical skin composition of claim 1, wherein the composition is a soothing massage body and skin product.

9. The topical skin composition of claim 1, wherein the composition is a topical method of treating the body and skin, the said composition soothes, massages, improves and enhances said body and skin.

10. The method of claim 1, wherein each of said extracts are obtained from the aloe vera plants, and the cactus plants, respectively.
